(19) United States
(12) Patent Application Publication
(10) Pub. No.: US 2008/0243197 A1
(43) Pub. Date: Oct. 2, 2008

(54) Therapeutic applicator system for applying light, sound and/or magnetism

(76) Inventor: Thomas Bove, Greenacres, WA (US)

Correspondence Address: WELLS ST. JOHN P.S., 601 W. FIRST AVENUE, SUITE 1300, SPOKANE, WA, 99201, US

(21) Appl. No.: 11/732,135

(22) Filed: Apr. 2, 2007

(51) Int. Cl. A61N 1/00

(52) U.S. Cl. 607/2; 601/15; 601/17; 601/22; 601/78

(57) ABSTRACT

This invention includes embodiments which combined the application or simultaneous application of magnetism, light waves and sound for a therapeutic effect, all through one applicator. The controller may be programmed or configured to provide relative timing of the application of the magnetism, light waves and sound relative to one another and in an intermittent way for a desired therapeutic effect. The applicator may also provide embodiments wherein one or more of magnetism, light waves and sound may be utilized to prepare the recipient to be a better recipient of one or more applications of magnetism, light waves and sound.

### CROSS REFERENCE TO RELATED APPLICATION

This application does not claim priority from any other application.

### TECHNICAL FIELD

This invention relates to an applicator system for the application of light, sound and magnetism to mammals for a therapeutic effect.

### BACKGROUND OF THE INVENTION

Modern medicine tends to attempt to solve health and related issues through chemical or surgical intervention. In some of these administrations of chemical intervention or drugs, they have been designed to target an enzyme system within the body or to create a localized immune system response so that the body's immune system actually solves the problem. Drugs contain side effects, however, and in some cases the benefit of drugs does not always outweigh the side effects. This invention is meant as an alternative, not a replacement, to some of the more traditional modern medicine interventions, and presents an alternative or holistic approach to healing in therapy.

It is an object of this invention to provide an applicator system for providing a therapeutic combination of light waves, sound waves and/or magnetism.

While the invention was motivated in addressing some objectives, it is in no way so limited. The invention is only limited by the accompanying claims as literally worded, without interpretative or other limiting reference to the specification, and in accordance with the doctrine of equivalents. Other objects, features, and advantages of this invention will appear from the specification, claims, and accompanying drawings which form a part hereof. In carrying out the objects of this invention, it is to be understood that its essential features are susceptible to change in design and structural arrangement, with only one practical and preferred embodiment being illustrated in the accompanying drawings, as required.

### BRIEF DESCRIPTION OF THE DRAWINGS

Preferred embodiments of the invention are described below with reference to the following accompanying drawings.

FIG. 1 is a schematic diagram illustrating the application of light waves to a cell which includes sodium chloride molecules;

FIG. 2 is a front elevation view of one aspect and of an applicator system contemplated by this invention;

FIG. 3 is a perspective view of an aspect of the invention, showing a cylindrical housing which may be utilized in the application of light;

FIG. 4 is a top view schematic representation of the aspect of the invention shown in FIG. 2;

FIG. 5 is an exploded schematic representation of an embodiment of an applicator system contemplated by this invention;

FIG. 6 is a schematic diagram illustrating the use and application of one embodiment of this invention;

FIG. 7 is an example of one aspect of the invention in a hand held applicator configuration; and

FIG. 8 is an elevation view of one aspect of the invention as utilized to impart electromagnetic waves via a foot pedestal arrangement.

### DETAILED DESCRIPTION OF THE PREFERRED EMBODIMENTS

Many of the fastening, connection, manufacturing and other means and components utilized in this invention are widely known and used in the field of the invention described, and their exact nature or type is not necessary for an understanding and use of the invention by a person skilled in the art or science; therefore, they will not be discussed in significant detail. Furthermore, the various components shown or described herein for any specific application of this invention can be varied or altered as anticipated by this invention and the practice of a specific application or embodiment of any element may already be widely known or used in the art or by persons skilled in the art or science; therefore, each will not be discussed in significant detail.

The terms a, an, and the as used in the claims herein are used in conformance with long-standing claim drafting practice and not in a limiting way. Unless specifically set forth herein, the terms a, an, and the are not limited to one of such elements, but instead mean at least one.

Electromagnetic waves exist across a wide range of frequencies, and a continuum within this range of frequencies may generally be referred to as the electromagnetic spectrum. The continuous range of frequencies is normally divided up into categories or labels based upon how those waves interact with other matter. These waves have varying wavelengths, with the longer wavelength and lower frequency regions being in the radio frequency band. Moving through the spectrum to shorter wavelength and higher frequency electromagnetic waves, there are also microwave frequencies, infrared rays or frequencies, visible arrays, ultraviolet rays, X-rays and gamma rays. The range of wavelengths and frequencies that are visible is actually very small in comparison to the entire electromagnetic spectrum. People generally use the term light to refer to that range of electromagnetic wave which stimulates their eyes, although it is not so limited. The visible light range is generally those wavelengths from approximately 700 nanometers (nm) to approximately four hundred (400) nm. In the visible light wave range, a wavelength generally represents a particular color because when that wavelength strikes the retina of our eyes, we perceive the color represented by that wavelength. And when a light wave of a particular frequency encounters an object, such as the human body, it either reflects the light wave, absorbs the light wave or some combination of both. Some objects selectively reflect, transmit and/or absorb certain frequencies of light.

It is generally known that atoms and molecules contain electrons, and the electrons have a tendency to interact with the atoms and molecules at a particular vibration frequency or range of frequencies, similar to a tuning fork or musical instruments. The electrons of atoms have a natural frequency at which they vibrate. When waves and objects at different frequencies interact, one may affect the natural vibration or wavelength of the other and cause a change. In some cases if a light wave with the same natural frequency of an electron strikes in object containing molecules or atoms with electrons of that natural frequency, those electrons will tend to absorb the energy of the light wave and vibrate differently or transform the energy from that electron into vibrational energy or motion. The electrons do not only have natural vibration or frequency with respect to the atom or molecule around which it is positioned, but also affect neighboring or adjacent atoms and their electrons. The effects on neighboring atoms may result in the conversion of the light wave to vibrational energy and then to thermal energy. The absorption of a light wave by an object and the effect of said absorption will tend to vary based upon the natural frequency of vibration of the electrons within an object relative to the frequency of the light wave being absorbed. Different molecules or atoms have different natural frequencies of vibration and therefore since they tend to absorb light waves with similar frequencies, they will selectively absorb different frequencies in the visible light range.

When a light wave strikes an object in which the natural frequency of vibration of the electrons is sufficiently different, the electrons may temporarily vibrate, but since they are out of frequency with the light wave received, the energy may behave as a light wave or be reflected. If the object being struck is what we generally refer to as transparent, then the vibrations of the electrons will be passed through the bulk of the material and emitted or re-emitted on the opposite side of the object, such as glass. If the material does not allow the passage of the light wave or the vibration from atom to atom, but instead reflects the energy wave out generally in the direction it came, that is a reflection thereof. The color that we perceive an object to be is largely dependent upon the way that it interacts with light waves that strike it and whether it transmits those light waves through the object or reflects them back. Therefore, the color of the object is not actually within the object itself, but instead the apparent color of an object is indicated by the way upon which it interacts when light hits it, and we then perceive the reflection of those light waves in the color range and perceive the object to be of a particular color.

Since different atoms and molecules within the body may have different natural frequencies, including healthy cells and unhealthy cells or unhealthy material within the body, an opportunity exists for the utilization of light waves throughout the electromagnetic spectrum to match the natural frequencies of the targeted atoms and molecules. For instance, different organs within the body may be associated with different light wave frequencies or different colors, and this may be referred to as the natural frequency of cells or atoms within that organ.

This invention combines energy and energy frequencies of light, sound, and magnetism, and allows a synergistic or integrated instrument to be tuned to specific desired wavelengths and these wavelengths may be matched to natural frequencies within the body to enhance cells of one or more natural frequencies since the wavelengths that will be transmitted and received by those cells will generally coincide with the natural frequency of those cells, atoms and/or molecules. Applying the desired frequency will result in the absorption of the light wave energy by those cells to which those light waves are preferably directed and its conversion to other forms of energy, including with a therapeutic effect. Those of ordinary skill in the art can see how the ability to apply more and different energy at different frequencies can enhance the ability to apply therapeutic solutions to an individual or mammal.

The simultaneous application of energy at different frequency levels through one applicator therefore may provide an infinite range of healing solutions, including through the intermittent application of one or all of the frequencies to create additional fluxes or changes within the cells.

The applicator surface may be made from a medical grade silicon flexible resin, to better facilitate the transmission of all the energy waves and frequencies desired to be applied. Silicone is generally used for both comfort reasons and functionality reasons and may also in and of itself further facilitate the generation or transmission of energy to static ions. If for instance silicone is used as the material at the surface interface, the silicone material may freely exchange static ions on the surface of the skin of the person to which the applicator is being applied and thereby increase blood circulation to the injured area.

The simultaneous application of sound with other forms of electrons in electromagnetic waves (such as light) to the targeted area may be accomplished in any one of a number of different ways as would be known by those of ordinary skill in the art. In this case, sound for example, may be generated through the use of a coil or series of turns from copper wire, or also by utilizing known speaker technology. The nature of the generation of sound and speaker technology is generally known and will not therefore be discussed in significant detail herein. For instance if a coil arrangement is utilized, the coil may be suspended by different mechanical methods and then surrounded by a permanent magnet. The coil will receive a sinusoidal wave of a particular frequency through the windings and cause pulsations to occur as it interacts with the permanent magnet. The silicone membrane may move with the coil pulsations. A high speed processor may be utilized in embodiments of this invention to generate the smooth sign wave signals at any one of a number of different frequencies, such as at approximately 60,000 segments per sine wave. The digital sine wave produced by a microprocessor may then be fed into a digital to analog converter, and the output of the digital to analog converter is connected to an amplifier. The amplitude of the amplifier may also be adjustable through variable resistance mechanisms. A memory circuit may then be utilized to store any number of different frequency combinations to apply a different frequency or combination of frequencies for a given application. For instance, the varying frequencies of a particular song or melody of a song may be stored in the memory circuit and utilized to produce the desired vibration and dynamic vibration frequencies on the surface of the applicator.

The light application portion of this invention may be generated by any one of a number of different ways. For instance the light source may be generated through the use of light emitting diodes (“LED'S”). LED's are currently utilized in a variety of medical applications, and light from LED's may replenish the energy supply of cells in the body if delivered or applied at the proper frequency to be absorbed by the targeted cell. If a device such as this invention has the ability to vary the frequency and provide numerous frequencies of different ranges, the range of cells that can be positively affected or therapeutically affected by such a device should greatly increase. The various figures provided with this application as well as the discussion relative to the figures below, show an embodiment of the light generating and transmitting portion of this invention.

In practicing embodiments of this invention it will be appreciated that light waves of different frequencies may be emitted through filters and lenses that isolate light of a particular frequency or color for application. Therefore the emission source of the light wave need not necessarily be a specific wavelength desired to be applied, but instead may include a wavelength or be alterable to a desired wavelength or frequency.

In some embodiments of this invention, a unique combination may achieve a modulating effect for the LED lights, by utilizing the sound waves to trigger a timing circuit which results in a smooth ripple effect that is synchronized with the sound being simultaneously generated and applied. The light waves of predetermined frequencies (which may be transmitted through concentric rings of light) may then be applied in rhythm with the sound waves being simultaneously applied, all controlled by a programmable microprocessor. The microprocessor may also be utilized to set the timing interval for the rays of light or different light waves so that any desired on-off sequence for the light waves and light waves may be achieved to apply electromagnetic waves intermittently. As the sound waves are created, the sinusoidal wave patterns of the audio signals may be received and analyzed, and a timing interval circuit therefore tuned to the sound waves to have a coordinated application of sound waves and light waves. A modulating effect may for instance utilize the frequency pattern of the audio waves as a carrier for the light energy.

The use of the combination of sound waves with light waves in some embodiments of this invention further facilitates the ability to first affect the vibration of atoms or electrons in cells to better receive light waves of the frequencies which may be provided thereafter, which may be utilized to enhance or amplify the therapeutic effect of this applicator system. New combinations of sound frequencies and light frequencies may be utilized to result in greater absorption of light energy or light photon energy to cells in the body.

A photon vibratory affect may excite the ions in the body on a molecular level. Ions such as sodium chloride have more than one atom. These types of molecules generally have had a minimum frequency of vibration and are easily excited by light and will vibrate adjacent molecules. This motion in physics terms may be referred to as a phononic vibratory effect. Ionic molecules correspond to a mode of vibration when positive and negative ions swing against each other at adjacent lattice sites, creating a time varying vibration.

A time variance circuit in this device divides the interval of the sound wave to a vibration that pushes, or excites, and then returns the molecules in the tissue to a relaxed state. This vibration is believed to cause molecular motion in cellular tissue helping to realign damaged cells and promote healing. As light excites these ionic molecules, a burst of sound energy excites the very atoms that comprise the molecules. Thus the combination of light and sound is created simultaneously in unison and synchronization. This is beneficial in areas where there is an energy crisis in the cells. As the light waves are triggered or set to the frequency of sound the energy supply of the cells has a greater potential to be replenished.

In some embodiments of this invention, a provision may be made to house a permanent magnet just underneath the surface of the applicator and within the silicone membrane. It will be appreciated by those of ordinary skill in the art, even though one permanent magnet may be referred to herein, one or more such magnets of different magnetism may also be utilized, depending on the desired polarity and application. The silicone is designed to pulsate with a vertical motion relative to the application service and the skin of the recipient. The vertical motion may be accomplished in some embodiments of this invention by using a copper coil that is attached to the silicone membrane. As sound waves are electrically signaled into the coil, mechanical pulsations occur. This way the mechanical pulsations produce a permanent static magnet in motion to the frequency of the sound waves applied.

As Albert Einstein indicated, when the motion of a magnet occurs, there arises a certain and definite energy field. This particular energy field has the ability to move electrons and create current potentials. This particular energy field is utilized in aspects of this invention at the surface of the device. The magnet is ultimately pulsating to the frequency of sound as the sound carries the mechanical pulsations of the silicone membrane that houses the magnet.

Some embodiments of this invention may therefore provide a new application which is the simultaneous application of energy and the frequency of energy in light waves, sound waves and magnetism, all working together in a single application system, which may then be utilized in combination for the desired therapeutic effect. Furthermore, by utilizing a wider variety of energies simultaneously and in sequence, more cells of varying or slightly varying natural frequencies may be positively affected, or may be therapeutically affected by the overall applicator system.

Embodiments of this invention may provide a threefold vibratory affect within the cells in the recipient. Light photons producing a phonon vibratory affect on ionic molecules may be the first; sound producing a phonon like effect on atoms may be the second; and magnetism producing a movement of the electrons may be combined for the desired therapeutic effect may be the third. These can occur in any order. This threefold vibration in effect may also be utilized to impose a push and a relaxation effect on the ions, atoms and electrons that make up our cellular tissue. With the understanding that we are comprised of atoms, and every atom has a relationship to the neighboring atoms, and with the further understanding that the Van der Waal's forces that hold the position or determine the position of atoms to neighboring atoms may be disrupted when illness or injury takes place, aspects of this invention including the applicator embodiment can help reposition or more normally position the atoms that are affected by the injury or illness.

This invention includes new combinations of energies, lights, sounds and magnetism in some embodiments of this invention, which may combine this energy in a synergistic instrument tuned to specific wavelengths. The instrument may enhance the normal body function of cells.

Sound therapy to help support and initiate the body's natural healing process is becoming more and more known for its therapeutic, healing and health benefits. In combining sound therapy with others, the sound wave may be utilized to vibrate or move the magnets and consequently the magnetic field so that magnetism therapy, with a vibrating magnet for instance, may be imparted on the recipient of the frequency and changing frequency of the sound or music being utilized.

The terms “magnet” and “magnetic material” may include any type or kind of magnet or magnetic material with no one in particular being required to practice this invention. By way of providing some, but not an exhaustive list of examples, this may include without limitation, permanent magnets, magnetic materials which create a changing magnetic field, ferromagnetic components, and others. The magnets or magnetic material may be fluxing, fixed, moving or otherwise, within the contemplation of this invention, and may create pulsed, changing, fluxing, modulating, and/or fixed/constant magnetic, waved or energy fields (as a few examples) within the contemplation of this invention. They may including alternating poles, north poles, south poles, or combinations thereof, and different shapes of the magnets and magnetic fields, all within the same magnet or magnetic layer, within the contemplation of this invention.

It will be appreciated by those of ordinary skill in the art that a device and method such as this, for the application of waves for various treatments can be used in any one of a number of different ways, with no one in particular being required to practice this invention. For instance, this invention may be used or attempted in the treatment of scar tissue, inflammation and the reduction of pain, as some of the possible examples of uses or embodiments for applications. The invention can be used to apply electromagnetic waves to something typically benefited by one of the therapies, but which may be enhanced by the simultaneous application of two or three of the therapies.

One of the components of this invention is a layer of silicone or a silicone module. Silicone is generally any one of various polymeric organic silicon compounds such as oils, greases, or plastics and used for water resistant and heat resistant lubrications, varnishes, binders, and as electric insulators. Silicone may come in many different forms, such as a rubber form made from silicon elastomers, which may be noted for its retention of flexibility, resilience and tensile strength over a wide temperature range.

In this case, a silicone layer or module, depending on the application, is designed to mechanically or operatively connect to a coil that can pulsate at any one of a number of different frequencies, or to vibrate or pulsate at multiple frequencies concurrently within the coil. The electrical coil is generally suspended and surrounded by a magnet or what may be also referred to as a permanent magnet, because it is generally preferred to fix the permanent magnet relative to a framework of the applicator.

Although a coil is generally a series of loops or a number of turns of wire, especially in a spiral form, for electromagnetic affect or for providing electrical resistance, it will be appreciated by those of ordinary skill in the art that other devices may be utilized which are technically not coils within the contemplation of this invention. Although the coil may be the preferred way to provide the recipient waves and to initiate vibration, there may be any one of a number of other devices which may be similarly used, although a wire coil is the preferred device. The coil may be suspended by any one of a number of different means, and then surrounded by a permanent magnet.

Generally as the coil receives sine wave frequencies through the windings, it causes pulsations to occur and the silicone module moves with the coil pulsations. The silicone module system may be configured to receive any shape and number of magnets by creating pockets within the geometry of the module or framework. The coil may receive sine wave frequencies from any one of a number of different sources or wave generators, such as a high speed microprocessor in some embodiments contemplated by this invention. The high speed microprocessor may be utilized to generate and transmit to the coil different digital, analog and sinusoidal frequencies or waves.

An interval circuit may also be in series with the output of the amplifier to control a time delay between pulses, or between frequencies or waves being transmitted.

It is a mechanical linkage or positional relationship between the silicone layer and the frequency driven coil that creates some of the benefits, which are believed to result from the use of the invention. The invention also has applications relating to methods utilizing silicone in conjunction with static magnets, which are connected to, mounted to or positioned relative to the applicator framework.

In past studies, experiments and uses of patches of silicone, the treatment of scar tissue has been accomplished over various areas. There have been numerous scientific studies regarding the efficacy of silicone sheets on scar tissue, and some of the scientific studies suggest that silicone works by forming a static electrical field between the silicone sheet and the skin of the person to whom the applicator is being applied. In the case of scar tissue, a smoothing and flattening affect has been shown to occur with an increase in blood circulation to produce an improved skin tone.

Other studies also suggest that static magnets can alter the behavior of nerve tissues, such as by the reduction of chronic pain sensations. It has been theorized that magnetism can suppress the actual potential firings of nerve impulses in humans, and thereby control or suppress pain and other effects of nerve impulses. Some of the studies referred to, test electromagnetic fields in and around the skin, and have indicated that the electromagnetism may also relieve pain by selectively increasing the excitability of large nerve fibers within the body, which then may block the pain gate for pain to be transmitted through nerves to the brain.

It will also be appreciated by those of ordinary skill in the art that the invention incorporates a silicone module containing pockets that can receive any shape, number and style of static magnets. The silicone module is designed to pulsate at any desired frequency or combination of frequencies. When the static magnets move, static magnetic fields simulate an electromagnetic field. The electromagnetism may be defined in part as a magnetic field that is coupled with electro sine wave paths. Embodiments of this invention utilize the mechanical pulsations of static magnets to achieve an infinitely adjustable pulsing magnetic field.

As stated above, silicone is one of the best known conductors of static electricity, and in some aspects of this invention, as the silicone module pulsates, an increase in static impulses is established. Many scientists have theorized that static electricity has a beneficial and profound healing affect on the human body.

Aspects of this invention further include a combination of a silicone layer for direct or near application to the skin, magnetism and a frequency which is imparted on the coil to develop and treat scar tissue, pain, inflammation, discomfort and skin tone related issues.

A microprocessor with memory storage is also utilized to create multiple sine wave frequencies into one output. In certain embodiments or aspects of this invention, the memory storage may be used such that a combination of frequencies can be placed into one input and the particular frequencies or wave pattern of any one frequency within the combination can be altered as desired.

The pulsations of the silicone module or applicator can be any one of a number of different pulsation rates from, for example, twenty (20) waves per second to twenty thousand (20,000) waves per second or hertz. It will also be appreciated by those of ordinary skill in the art that as the multiple frequencies are mixed into one output, the amount of resistance or friction is increased, which causes both rapid and slower pulses within the same wavelength. The exchange between both negative and positive electrons along the surface body of atoms is also increased during a particular wavelength.

The term “therapeutic” is also used herein to cover and include any such wave or energy source which has a therapeutic, health care or biological affect on a recipient, including a magnetic field, a static field, an electric field, infrared waves or any others with a therapeutic or health care affect. For example, it may later be determined that wave forms or energy anywhere in the electromagnetic spectrum or sound wave spectrum provide a therapeutic or health care affect to the recipient, which are intended to be included herein.

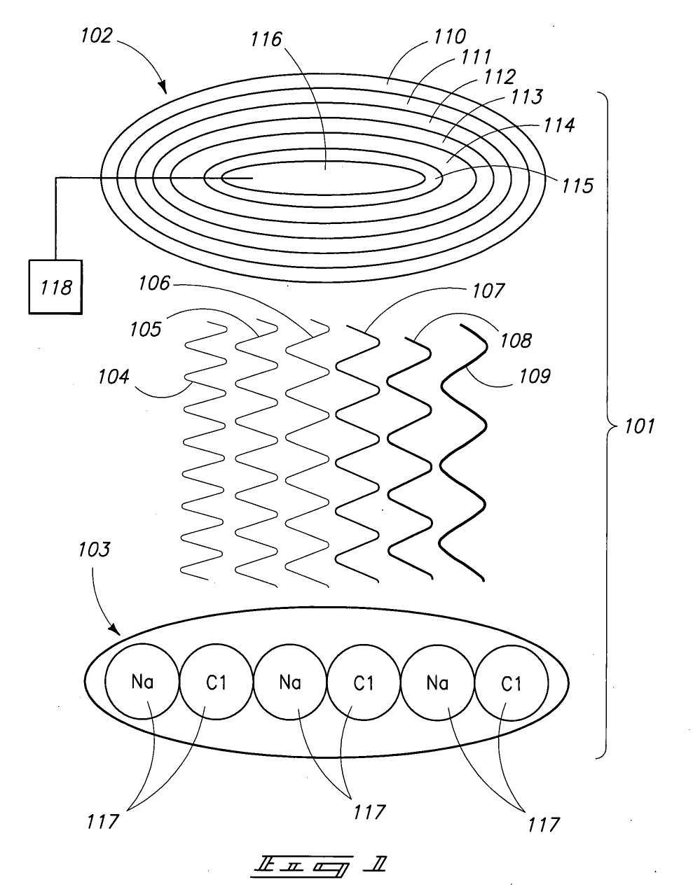

FIG. 1 is a schematic diagram illustrating the application of light waves to a cell which includes sodium chloride molecules therein. It will be appreciated by those of ordinary skill in the art that the application of any of the energies referred to herein, including light, magnetism or an electromagnetic field, and/or sound may be applied to different types of cells or components in the body for the desired or therapeutic effect, with no one in particular being required to practice this invention.

FIG. 1 shows light applicator 102 with a plurality of sources of light waves, represented by first light wave 104, second light wave 105, third light wave 106, fourth light wave 107, fifth light wave 108 and sixth light wave 109. Center 116 may also be utilized to locate sound, magnetism or light sources of therapeutic application. FIG. 1 further shows controller 118 operatively attached to light applicator 102, and which may be utilized to control all of the application, timing, frequency and other parameters desired for the therapeutic application or use of the applicator.

The light waves are shown in a representative way, but may represent one of a number of different colors or frequencies and may further represent the full range of color from the electromagnetic spectrum. Since the wavelength of each color is a different frequency, the slower frequencies may be initiated or energized first. It will also be appreciated by those of ordinary skill in the art that if any one of a number of different shapes of the applicator, i.e. whether it be elliptical, circular, a polygon or any other shape, with no one in particular being required to practice the invention. An elliptical or circular shape may for example be chosen for direct application to skin, whereas polygonal or other shapes may be used for other applications, with no particular shape being required to practice this invention or for any specific application, all within the contemplation of this invention.

Furthermore, this invention does not require that the light waves be applied simultaneously, but instead they may be simultaneously applied in a procession or randomly, depending on the specific application. This application might illuminate each light source in a time cycle either relative to the other sources of colors or frequencies of light, or relative to the electromagnetic field being applied and/or the sound waves being applied. It will further be appreciated by those of ordinary skill in the art that the application of one or more of the light waves may be controlled and timed to be intermittent, such as intermittently interjected with a strobe process, which would have the effect (together or separate from other therapeutic applications) of energizing, exciting or affect the molecules, then allowing the molecules to return to a relaxed or normal state, and then again exciting or affect in the same molecules. This invention brings together light, sound and/or magnetism for any one of a number of different therapeutic applications. In some applications where a pressure wave is exerted by one of the therapeutic applicators, the metabolic system of the human body may be duplicated or it may be offset to achieve the desired therapeutic results.

In some embodiments of this invention, this applicator may utilize light, magnetism and sound to reposition atoms, molecules and/or electrons within the human body for therapeutic applications. These applications may be designed to achieve a desired therapeutic or healing response by the body or by the bodies in the new system, thereby achieving the desired result through use of the body itself.

FIG. 1 is a schematically exploded view for illustration purposes and shows the light applicator 102 space apart from the representative cell 103; however it will be appreciated that the applicator may be touching or spaced apart at any one of a number of different distances, depending on the application and therapeutic result desired. FIG. 1 shows a representative cell which includes sodium chloride molecules 117 which, in some applications of this invention, may be affected by the applicator through the light waves, magnetism or an electromagnetic field (which may include an electric field) or the sound applied.

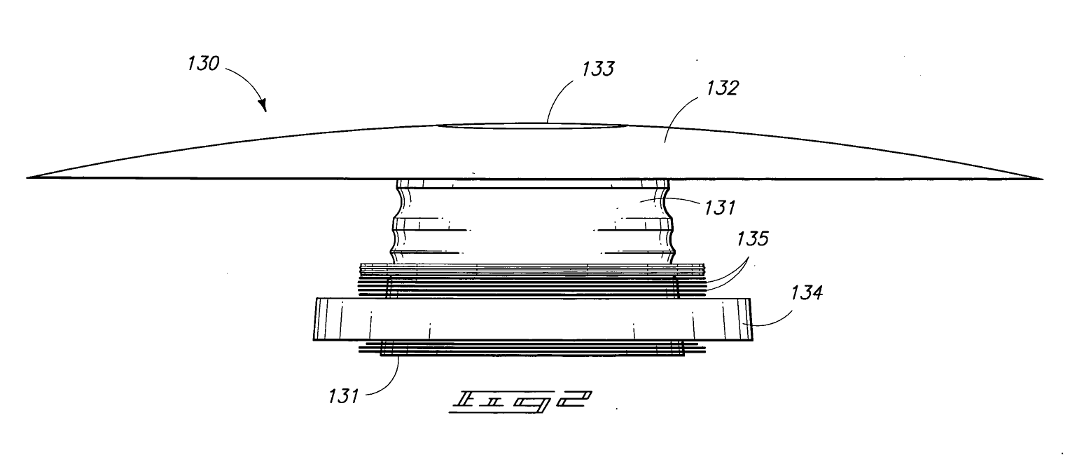

FIG. 2 is a front elevation view of another embodiment of an applicator system 130 contemplated by this invention, showing static permanent magnet 133, surrounding magnet 134, copper coil 135 around body 131, wherein the applicator interface 132 may include a translucent cover which may for instance be a clear silicon membrane molded to the desired shape. The silicon would be clear or translucent to allow the light waves from the light applicator 102 shown in FIG. 1, to be transmitted there-through. Again, any one or more of a number of different colors or color frequencies may be used depending upon the number of cavities of opaque resin located below the silicon membrane. A static permanent magnet 133 is embedded within the membrane to provide part or all of the electromagnetic field being applied to the recipient of this therapeutic system.

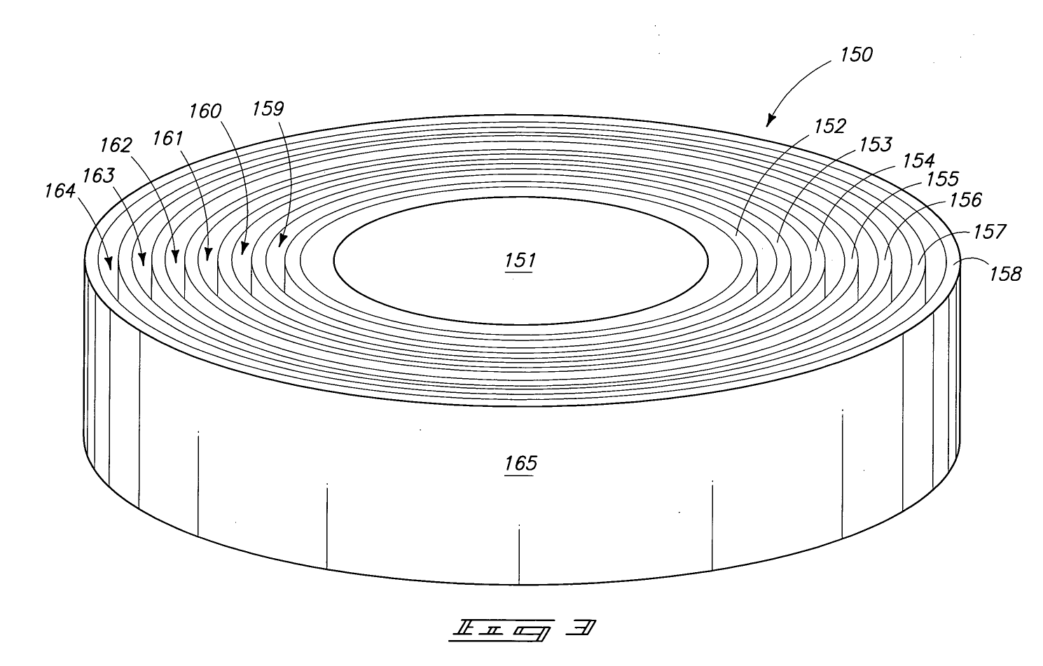

FIG. 3 is a perspective view of an embodiment of the invention, showing a cylindrical housing and which may be utilized in the application of light to an intended recipient. FIG. 3 illustrates a cylindrical housing that may be molded from an opaque thermoplastic resin, with translucent lenses that may molded in two separate various color spectrums. This light housing 150 may be located underneath the clear silicon sound module as reflected in FIG. 2.

FIG. 3 illustrates housing outer wall 165, center aperture 151, first light aperture 164, second light at the chair 163, third light aperture 162, fourth light aperture 161, fifth light aperture 160 and sixth light aperture 159, with the light apertures extending around center 151. Opaque walls 152, 153, 154, 155, 156, 157 and outer wall 158 serve to separate the light apertures is referenced above. The material may be made of any one of a number of different materials, with thermoplastic resin being one of the potential materials which may be utilized in some applications of this invention. Material may be chosen for any one of a number of different properties, including opaque characteristics, translucent characteristics, structural characteristics, filtering characteristics, and reflective characteristics, especially reflection on the surfaces and what may be desired to better facilitate the passage of light through the light aperture is between the opaque walls.

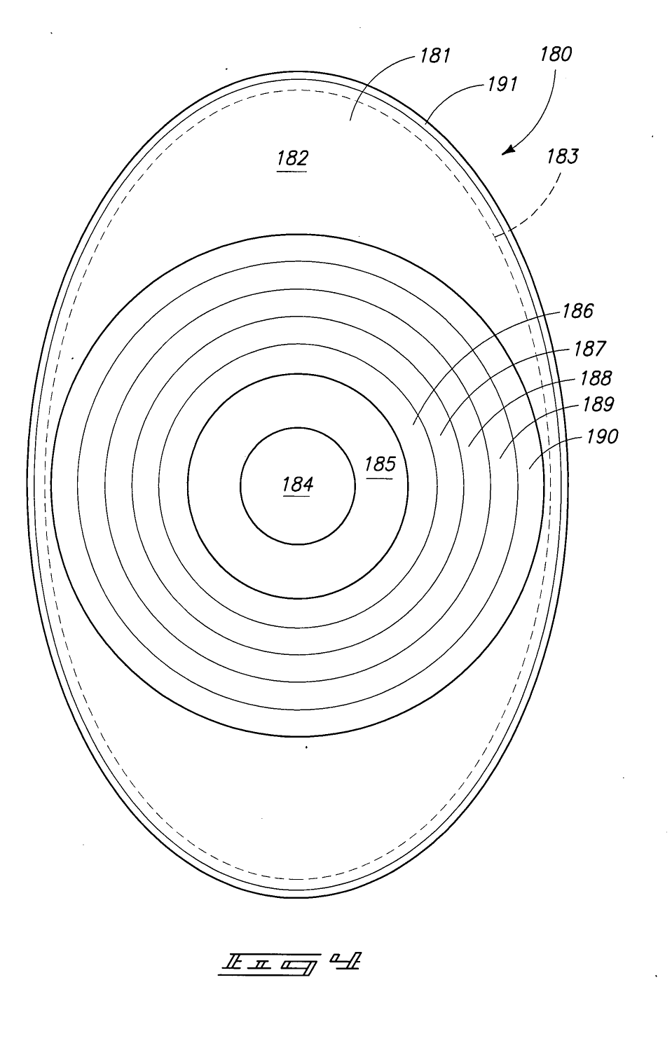

FIG. 4 is a top view schematic representation of one embodiment of an applicator contemplated by this invention. FIG. 4 shows an applicator 180 with applicator interface 181 including the top surface 182. A housing 183 is shown within the applicator interface 181, with the housing being similar to the example shown in FIG. 3 and further discussed above. This Figure schematically represents light passageways 185, 186, 187, 188, 189, and 190 which may, but need not, be constructed or configured similar to that shown in FIG. 3. Center 184 is shown in the middle, but may be in any location for the application of light, sound or magnetism, with no one specific location being required for all aspects or embodiments of the invention.

As sound waves are generated, a timing circuit control system may have a modulating effect on the rings of light, and any one in the number of timing arrangements may be programmed with the use of a microcontroller or other device, which may be located on a printed circuit board or remotely or within the applicator itself. If music is used as part of the sound application, the light might be programmed in the rhythm over the beat with the music. The sequence can be programmed to have a different interval time sequence so that each ring of light applied through the light apertures may be turned on or off at any desired time or at any desired sequence.

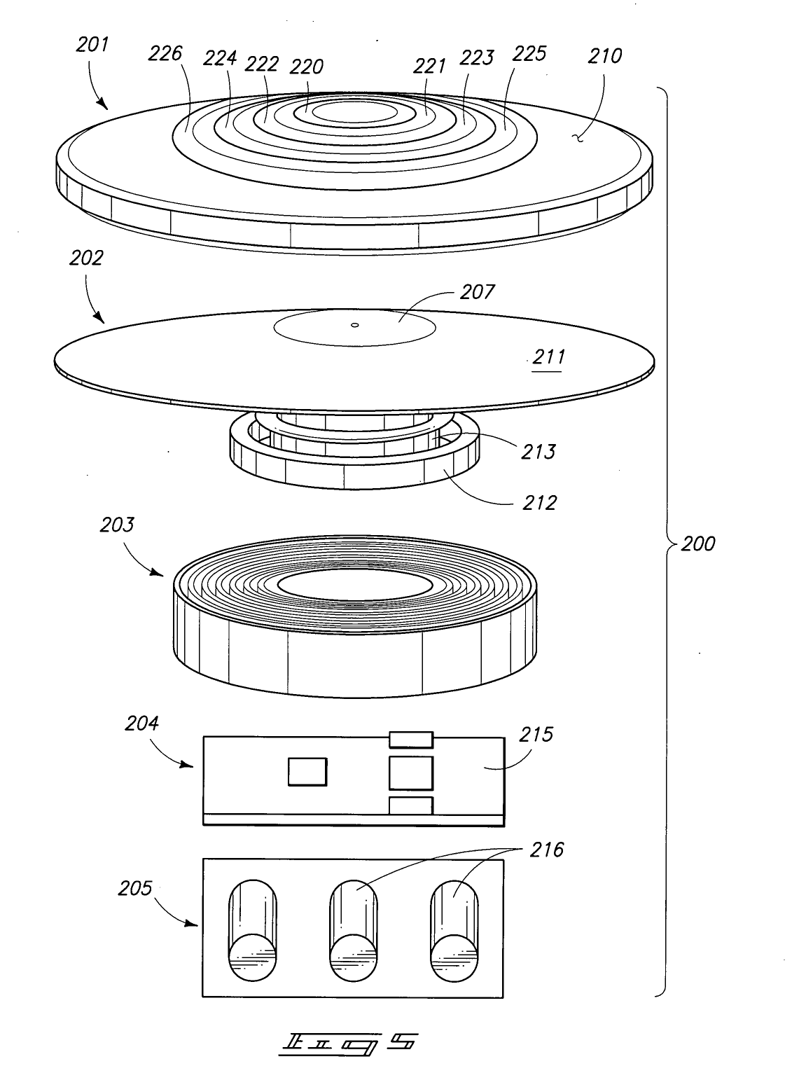

FIG. 5 is an exploded schematic representation 200 of one embodiment of an applicator system contemplated by this invention. FIG. 5 illustrates translucent lenses to 220, 221, 222, 223, 224, 225 and 226 on housing 201 with outer surface 210. The lenses may also be or include filtering elements in some aspects of this invention. FIG. 5 further shows applicator interface silicon flexible membrane with static permanent magnet 27 therein. The silicon flexible membrane is translucent as described with respect to FIG. 2 and FIG. 4. The light housing 203, coil ring magnet assembly 212 around body 213, control components 204, which may be a printed circuit board 215 with whatever control components are desired. This may be utilized to generate and transmit light at any one of a number of different frequencies, and may contain a microprocessor to time and sequence the application of light waves to the recipient. It will be appreciated by those of ordinary skill in the art that any one of a number of different light generators, filters and timing programs may be utilized in aspects of this invention, with no one in particular being required to practice this invention. It will also be appreciated that the intermittent application of light waves in different sequences and for varying times may be desired in applications of this invention, which can be readily programmed with the microprocessor and control components 204. FIG. 5 further shows power supply 205 which may be batteries 216 or any other source of power.

The control components 204 shown in FIG. 5 may also be utilized to receive or generate sound waves, such as music or other desired sound waves, and to transmit it through the coils to cause the magnet to vibrate to the sound waves. It will be appreciated by those of ordinary skill in the art that this harmonization of the vibration of the magnetic field and the fluxes created thereby, at the same frequencies as the sound waves and music, may achieve the desired therapeutic affect.

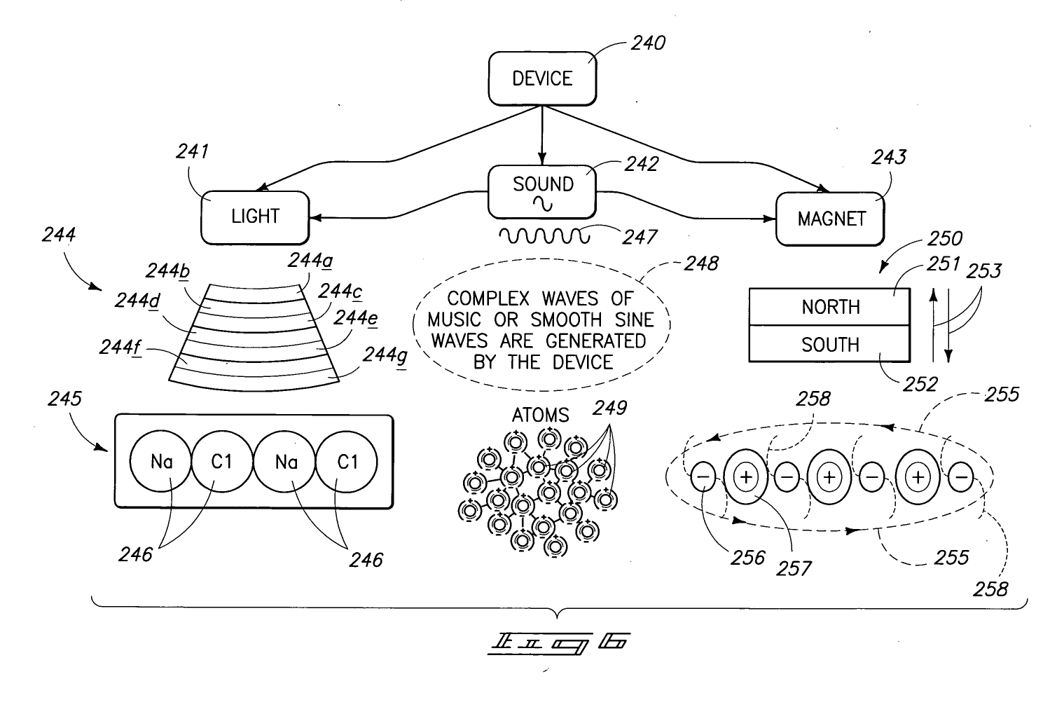

FIG. 6 is schematic diagram illustrating the use and application of one embodiment of this invention. FIG. 6 shows schematic representations of various components and attributes of some embodiments of this invention, including device 240 which is comprised of a light portion 241, a sound portion 242 and a magnet portion 243.

With respect to the light portion 241, schematic representation 244 illustrates how light of different frequencies and colors may be applied to a representative portion of a body 245, which may include sodium chloride 246 components within the body. There are generally ions or ion molecules within most living tissue including humans, and sodium chloride is one example of one which may be present in the human body. When exposed to light, these ion molecules such as sodium chloride tend to vibrate. As the light rays or light waves modulate, the molecules within the body vibrate and then relax. This motion may be accomplished in sequence with complex sound waves created by the device as explained below. In the first light wave 244a, second light wave 244b, third light wave 244c, fourth light wave 244d, fifth light wave 244e, sixth light wave 244f and seven light wave 244g, shown in a representative manner to show one aspect or embodiment that need be utilized in practicing this in the light portion 241 of this invention.

In addition to affecting specific cells and organs, different colors in the color spectrum are also associated with imparting different effects or therapeutic effects on the body. For instance the color violet may be utilized to soothe certain organs, relax muscles or calm the nervous system, whereas indigo may have a calming effect, or control other functions. Green for instance, which is toward the middle of the visible color spectrum, is calming and associated with balance as well as having positive therapeutic effects against bacterial infections or conditions. It will be appreciated by those of ordinary skill in the art that these additional potential therapeutic benefits may also be combined with the other attributes of this applicator for the desired results.

The sound portion 242 is represented by soundwave 247 and area 248 indicates that complex waves of music or smooth sound waves may be generated by the device and applied to atoms 249 or components of atoms within the body. Van der Waal's forces are sometimes referred to in defining or identifying the relationship between atoms with neighboring atoms. Gluons may establish the memory position of atoms to neighboring atoms. Smooth and harmonious sound waves may allow atoms to be shifted back to their original position before they were displaced through the frequency of the applied waves. A therapeutic application may be to move or align one electron in the atoms consistent with their original alignment or positioning, as compared to the excited or displaced position which created the need for the therapeutic application. The complex waveforms generated by the device may apply a pressure (or excite) and then a relaxation effect to the atoms. This would be in a vibrational manner and the vibratory effect is similar to the action of a tuning fork when this sound is applied.

The magnet portion 243 of this invention is schematically shown by the north pole 251 and south pole 252 representation of a magnetic field 250 with arrows 253 showing the magnetic forces. The schematic representation below the magnet 250 shows one aspect of how an electromagnetic field may impose forces on the intended recipient, showing a force field 255 with negative charge 256, positive charge 257, and in magnetic field indicators 258 reflecting an electromagnetic field generated. Generally, when a static permanent magnet is in motion, a certain energy field emerges from the transients or flux from the movements. This energy field has the ability to move electrons and create current potentials. As the static permanent magnet pulsates from the mechanical movements created by the electrical impulses of the complex soundwave from the device, a push and relaxation effect is exerted on any movable electrons within the body.

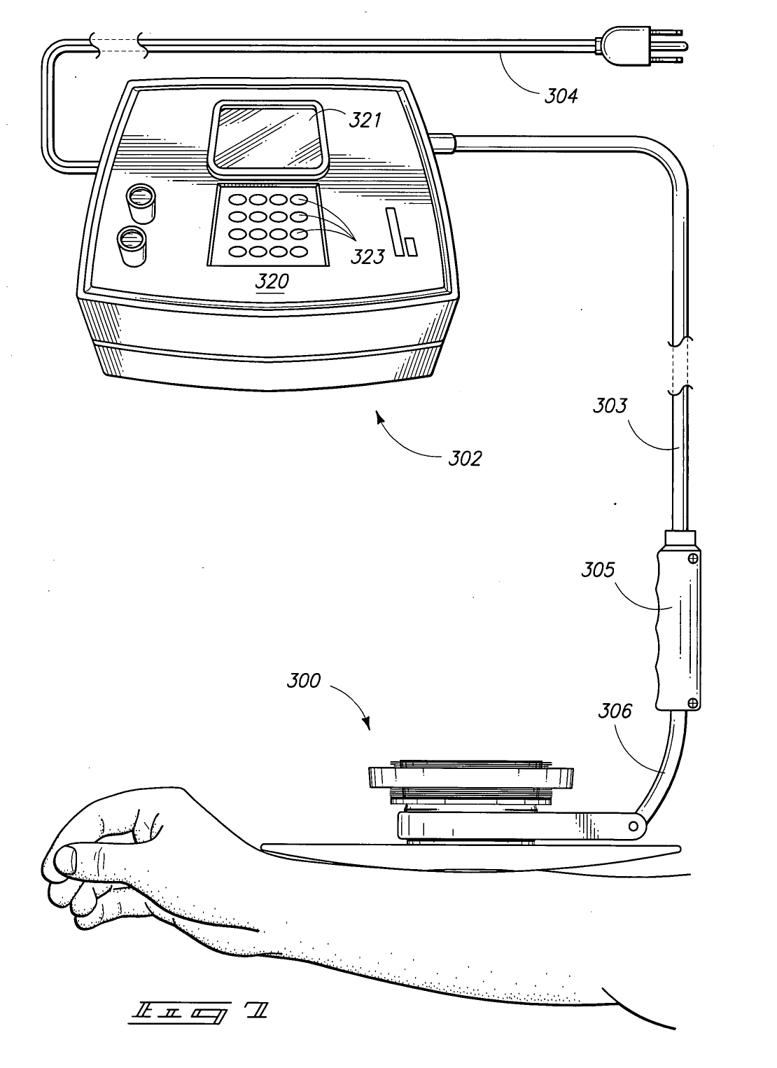

FIG. 7 is an example of one aspect of the invention in a hand held applicator configuration, illustrating hand held applicator 300, holding structure 306, with applicator 300 pivotally mounted to holding structure 306 for application to an arm for a therapeutic or desired effect. FIG. 7 also shows conductor 303 which may be a combination of conductors to facilitate the transmission of light, sound and/or electrical waves from generator 302. The generator housing 320 may be any one of a number of types of housings, including one of a number of types of controls or displays, such as keypad 323 and display 321. It will also be appreciated by those of ordinary skill in the art that this invention may use a separate or free-standing generator 302 as shown, or a more compact generator contained within the handle 305 and/or framework for the applicator itself, all within the scope of the invention and with no one in particular being required to practice this invention.

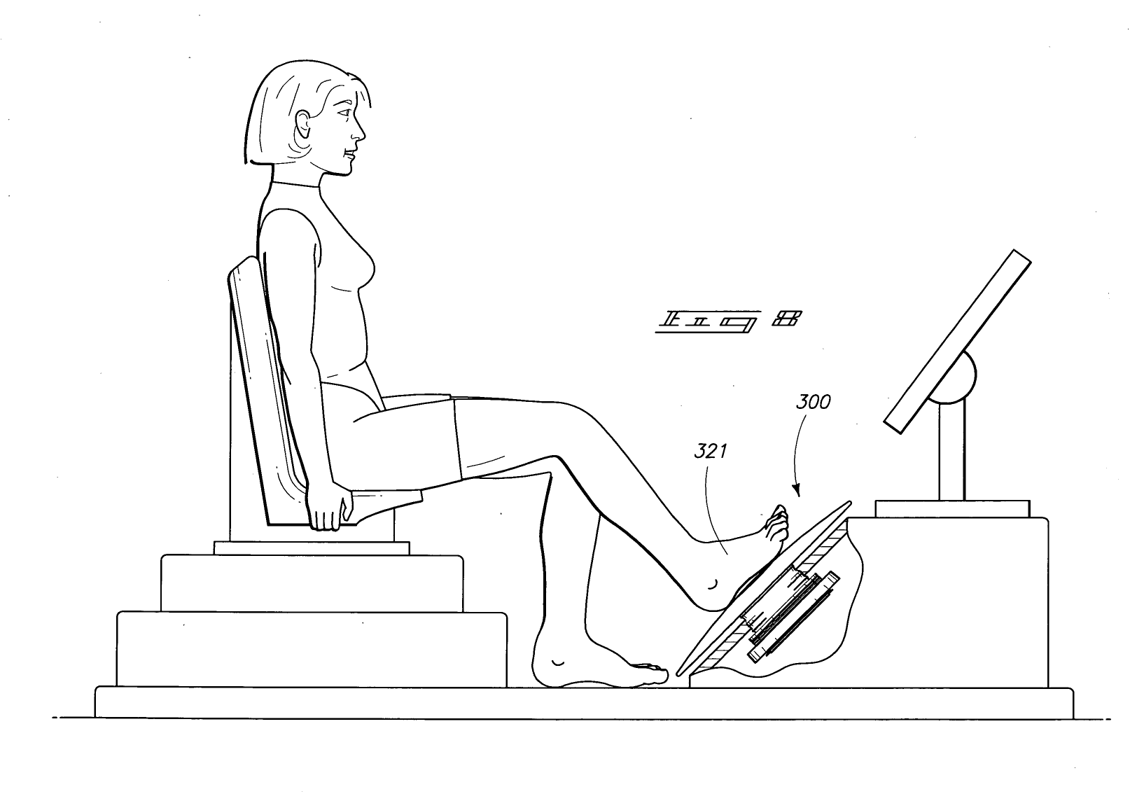

FIG. 8 is an elevation view of one aspect of the invention as utilized to impart electromagnetic waves via a foot pedestal 300 arrangement, illustrating a user's foot 321 on the application surface. Those of ordinary skill in the art will understand that there are numerous applications for different aspects of this invention with the hand-held applicator and the foot pedestal application 300 being merely two of numerous possible examples, with this invention not being limited to any one.

In order to illustrate an aspect of the invention, an exemplary application will be described. Human muscle fibers tend to have a frequency of vibration at approximately one-hundred eighty Hertz (Hz). Assuming for the example that a particular muscle fiber has a frequency mode of vibration of approximately one-hundred eighty Hz, the applicator may utilize multiple sine wave frequencies that are above and below this frequency mode of vibration. In the case of muscle fiber treatment, a setting may be at one-hundred-forty Hz, one-hundred-fifty-five Hz, one-hundred-eighty Hz, two-hundred-ten Hz and two-hundred-seventy-five Hz. These multiple frequencies when mixed together create a complex wave form, which may provide a mode of vibration and a mode of relaxation within a time interval. Since frequencies correspond to the reciprocal of time, the complex wave form will create moments of pressure and moments of relaxation at the given combination of frequency.

Each individual cellular tissue may also have its own resonating frequency mode of vibration. It is believed that this mode of vibration is directly related to a specific gravity and surface tension. Surface tension may be a measurement of the time it takes to crate an elastic movement with a given amount of energy. The frequency mode of vibration is at a higher level when the surface tension is greater.

In this application of this aspect of the invention, the concentric rings of light may work in a similar way, and it should be kept in mind that the device may utilize the full visible electromagnetic spectrum of energy. This measurement may for example be from a wavelength of approximately four-hundred nm to approximately six-hundred-fifty nm. It is believed that most molecules within the human body will vibrate at approximately a five-hundred nm wavelength. For the purpose of creating a mode of vibration and a mode of relaxation, a typical setting of the device will turn on the lower spectrum first or a period of two seconds or so, and then turning on each higher light wave at one second intervals, until the full visible spectrum is reached. The timing sequence will then proceed in the reverse order.

As will be appreciated by those of reasonable skill in the art, there are numerous embodiments to this invention, and variations of elements, components and combinations, which may be used, all within the scope of this invention.

For example, in one embodiment, an electromagnetic wave applicator for applying electromagnetic waves is disclosed, the wave applicator being comprised of: an applicator framework; a light wave applicator disposed relative to the applicator framework to emit light waves of at least one frequency on the skin of the mammal; a sound wave applicator disposed relative to the applicator framework to direct sound waves to the mammal; at least one magnet movably mounted relative to the framework; and an electrical coil mounted relative to the framework and positioned relative to the at least one magnet such that electrical signals through the electrical coil influence the movement of the at least one magnet, wherein the electrical coil is configured to receive at least one wave from a controller. Additional embodiments of the forenamed may include an electromagnetic wave applicator: which further includes at least one wave provided to the electrical coil is a sound wave provided by the sound wave applicator, thereby providing vibration of the at least one magnet at the same frequency as the sound waves; further wherein the applicator is configured as part of a pedestal configured to receive feet of a user; further comprising a silicone layer operatively connected to the wave applicator framework and configured for placement at or near the skin of a mammal; and/or further wherein the light wave applicator is configured to intermittently apply light waves to the mammal.

In another embodiment, an electromagnetic wave applicator for applying electromagnetic waves to a mammal is disclosed, in which the wave applicator includes: an applicator framework; a silicone layer operatively connected to the wave applicator framework and configured for placement at or near the skin of a mammal; at least one magnet mounted relative to the framework; an electrical coil movably mounted relative to the at least one magnet, wherein the electrical coil is configured to receive at least one wave and to thereby influence movement of the at least one magnet; a light wave applicator operatively attached to the applicator framework to emit a plurality of light waves of different frequencies onto the skin of the mammal; and a sound wave applicator disposed relative to the applicator framework to transmit sound waves to the mammal. Additional and further embodiments of the foregoing may include such an electromagnetic wave applicator and further: wherein the light wave applicator emit a plurality of concentric light waves of different frequencies onto the skin of the mammal; and/or further wherein the at least one wave provided to the electrical coil is a sound wave provided by the sound wave applicator, thereby providing vibration of the at least one magnet at the same frequency as the sound waves.

In another embodiment, an electromagnetic wave applicator for applying electromagnetic waves is disclosed, the wave applicator including: an applicator framework; at least one magnet mounted relative to the applicator framework, disposed to impart a magnetic field to a mammal; a light wave applicator disposed relative to the applicator framework to emit light waves of at least one frequency on the skin of the mammal; and a sound wave applicator disposed relative to the applicator framework to transmit sound waves to the mammal.

In yet another embodiment, an electromagnetic wave applicator for applying electromagnetic waves is disclosed, the wave applicator including: an applicator framework; at least one magnet mounted relative to the applicator framework, disposed to impart a magnetic field on a mammal; a light wave applicator disposed relative to the applicator framework to emit light waves of at least one frequency on the skin of the mammal and wherein the light wave applicator emit a plurality of concentric light waves of different frequencies onto the skin of the mammal; and a sound wave applicator disposed relative to the applicator framework to transmit sound waves to the mammal.

In a method embodiment of the invention, a method for applying a plurality of different electromagnetic waves to a mammal is disclosed, which includes: applying a vibrating magnetic field to a mammal; applying light waves of at lease one frequency to the mammal simultaneous to applying the vibrating magnetic field; and applying sound wave therapy simultaneous to applying the vibrating magnetic field. This method may further include vibrating the magnetic field at the same frequency as the sound wave frequency; and/or applying the light waves intermittently.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

What is claimed is:

1. An electromagnetic wave applicator for applying electromagnetic waves to a mammal, the wave applicator being comprised of:
an applicator framework;
a light wave applicator disposed relative to the applicator framework to emit light waves of at least one frequency on the skin of the mammal;
a sound wave applicator disposed relative to the applicator framework to direct sound waves to the mammal;
at least one magnet movably mounted relative to the framework; and
an electrical coil mounted relative to the framework and positioned relative to the at least one magnet such that electrical signals through the electrical coil influence the movement of the at least one magnet, wherein the electrical coil is configured to receive at least one wave from a controller.

2. An electromagnetic wave applicator as recited in claim 1, and further wherein the at least one wave provided to the electrical coil is a sound wave provided by the sound wave applicator, thereby providing vibration of the at least one magnet at the same frequency as the sound waves.

3. An electromagnetic wave applicator as recited in claim 1, and further wherein the applicator is configured as part of a pedestal configured to receive feet of a user.

4. An electromagnetic wave applicator as recited in claim 1, and further comprising a silicone layer operatively connected to the wave applicator framework and configured for placement at or near the skin of a mammal.

5. An electromagnetic wave applicator as recited in claim 1, and further wherein the light wave applicator is configured to intermittently apply light waves to the mammal.

6. An electromagnetic wave applicator for applying electromagnetic waves to a mammal, the wave applicator being comprised of:
an applicator framework;
a silicone layer operatively connected to the wave applicator framework and configured for placement at or near the skin of a mammal;
at least one magnet mounted relative to the framework;
an electrical coil movably mounted relative to the at least one magnet, wherein the electrical coil is configured to receive at least one wave and to thereby influence movement of the at least one magnet;
a light wave applicator operatively attached to the applicator framework to emit a plurality of light waves of different frequencies onto the skin of the mammal; and
a sound wave applicator disposed relative to the applicator framework to transmit sound waves to the mammal.

7. An electromagnetic wave applicator as recited in claim 6, and further wherein the light wave applicator emit a plurality of concentric light waves of different frequencies onto the skin of the mammal.

8. An electromagnetic wave applicator as recited in claim 6, and further wherein the at least one wave provided to the electrical coil is a sound wave provided by the sound wave applicator, thereby providing vibration of the at least one magnet at the same frequency as the sound waves.

9. An electromagnetic wave applicator for applying electromagnetic waves to a mammal, the wave applicator being comprised of:
an applicator framework;
at least one magnet mounted relative to the applicator framework, disposed to impart a magnetic field to a mammal;
a light wave applicator disposed relative to the applicator framework to emit light waves of at least one frequency on the skin of the mammal; and
a sound wave applicator disposed relative to the applicator framework to transmit sound waves to the mammal.

10. An electromagnetic wave applicator for applying electromagnetic waves to a mammal, the wave applicator being comprised of:
an applicator framework;
at least one magnet mounted relative to the applicator framework, disposed to impart a magnetic field on a mammal;
a light wave applicator disposed relative to the applicator framework to emit light waves of at least one frequency on the skin of the mammal and wherein the light wave applicator emit a plurality of concentric light waves of different frequencies onto the skin of the mammal; and
a sound wave applicator disposed relative to the applicator framework to transmit sound waves to the mammal.

11. A method for applying a plurality of different electromagnetic waves to a mammal comprising the following:
applying a vibrating magnetic field to a mammal;
applying light waves of at lease one frequency to the mammal simultaneous to applying the vibrating magnetic field; and
applying sound wave therapy simultaneous to applying the vibrating magnetic field.

12. A method for applying a plurality of different electromagnetic waves to a mammal as recited in claim 11, and further wherein the vibrating magnetic field is vibrated at the same frequency as the sound wave frequency.

13. A method for applying a plurality of different electromagnetic waves to a mammal as recited in claim 11, and further wherein the light waves are applied intermittently.
